(19) United States
(12) Patent Application Publication
(10) Pub. No.: US 2019/0381235 A1
(43) Pub. Date: Dec. 19, 2019

(54) VASCULAR ACCESS CATHETER WITH PROTECTABLE INLINE NEEDLE AND ASSOCIATED METHOD OF USE THEREOF

(71) Applicant: Aadi Innovations LLC, Columbus, OH (US)

(72) Inventor: Aaditya Shidham, Columbus, OH (US)

(21) Appl. No.: 16/299,775

(22) Filed: Mar. 12, 2019

Related U.S. Application Data
(63) Continuation-in-part of application No. 16/012,007, filed on Jun. 19, 2018.

(51) Int. Cl. A61M 1/36; A61M 25/00

(52) U.S. Cl. CPC A61M 1/3661; A61M 25/0097; A61M 1/3655

(57) ABSTRACT

Exemplary embodiments described herein are directed to a vascular access catheter device with an inline needle, the device having a catheter portion that is linearly displaceable relative to a needle portion, and vice versa, and to methods of performing hemodialysis using said device. Extension of the catheter portion relative to the needle portion allows the catheter of the catheter portion to cover the needle tip, thereby minimizing or eliminating the possibility that the needle may damage a fistula/graft or peripheral vessel of a patient. Subsequent retraction of the needle portion withdraws the needle into the catheter to protect the fistula/graft or peripheral vessel from possible damage. Because the needle still remains largely within the catheter after retraction, there is no risk of catheter collapse, kinking, etc., which could compromise blood flow during a hemodialysis or other vascular access procedure.

### CROSS-REFERENCE TO RELATED APPLICATIONS

This application is a continuation-in-part of U.S. application Ser. No. 16/012,007, filed on Jun. 19, 2018, which is hereby incorporated by reference as if fully recited herein.

### TECHNICAL FIELD

Exemplary embodiments described herein are directed to a vascular access catheter with an inline needle that is protectable by sliding displacement of the catheter.

### BACKGROUND

Worldwide there are currently about 2 million patients with End Stage Renal disease (ESRD). There were about 660,000 such patients in the United States as of 2013 according to the United States Renal Data System (USRDS). Out of those 660,000 ESRD patients, about 465,000 patients were receiving hemodialysis treatment, which is the process of removing extra fluids and toxins from the body and maintaining normal electrolyte levels by passing the patient's blood through a dialysis filter and subsequently returning the blood to the patient. Hemodialysis is typically performed in 3-4 hour sessions, three times per week.

Because hemodialysis involves extracting blood from the patient and returning the filtered blood to the patient, hemodialysis obviously requires repeated access to the arteriovenous system of the patient. A fistula or graft is commonly created in order to provide an effective vascular access point. An arteriovenous (AV) fistula is a surgically created direct connection of an artery to a vein, which becomes a permanent (but surgically reversible structure after a sufficient healing period). An AV graft, while similar to an AV fistula, employs a plastic tube to connect and artery to a vein. In either case, once the fistula/graft is ready to use, the fistula/graft provides the arteriovenous access required to remove and return a patient's blood during hemodialysis treatment. The proper function of a fistula/graft is critically important for most hemodialysis patients.

One of the most important steps in the hemodialysis process is cannulation of the fistula/graft. Two needles are typically inserted into a fistula/graft for arteriovenous access. One of the needles is used to direct blood from the patient through a connected tube to the dialysis filter of the dialysis machine, while the other needle is used to direct filtered blood from the dialysis machine through a connected tube back to the patient. Required blood flow through the needles may be on the order of 250-550 ml/min. To achieve this blood flow, the needles used are usually of large diameter (e.g., between 17 and 14 gauge).

In operation of most currently used hemodialysis needles, the skin of a patient is pierced by the tip of the dialysis needle, and the needle is further inserted until the tip also pierces the fistula/graft. Once the needle is properly placed in the fistula/graft, it is normally secured in place by taping it to the associated limb of the patient.

As should be obvious, the tip of a hemodialysis needle is sharp. Use of a typical hemodialysis needle generally results in the sharp tip of the needle floating in the fistula/graft. Consequently, any needle migration or movement of the limb of the patient in which the fistula/graft has been created, can cause the needle tip to damage the fistula/graft, such as by causing a counter puncture of the fistula/graft wall. Such a puncture can result in infiltration where blood leaks outside of the fistula/graft. Studies have reported an annual infiltration rate of about 5.2% of all hemodialysis treatments, which is a significant problem.

Because blood is typically flowing through the hemodialysis needles at rate of 250-550 ml/min, a significant amount of blood can accumulate in surrounding tissues and form a hematoma as a result of a counter puncture and associated infiltration. An infiltration can be very painful, and often times requires that the fistula/graft be allowed to heal before further use. Thus, a patient may either have to miss dialysis treatment for a period of time, or a temporary dialysis catheter may need to be placed in a central vein of the patient in order to permit continued dialysis until the fistula/graft heals. It is also possible that an infiltration can lead to permanent loss of use of the damaged fistula/graft.

It can be understood from the foregoing description that there is a heretofore unmet need for an improved device and method for providing hemodialysis arteriovenous access without fistula/graft damage. A similar need exists for providing peripheral vessel access without damage. Exemplary vascular access catheter devices described herein satisfy this need.

### SUMMARY

Exemplary vascular access catheter device embodiments described and shown herein are designed to provide hemodialysis arteriovenous access without fistula/graft damage, and may also be used to access peripheral vessels in non-dialysis patients. Generally speaking, exemplary vascular access catheter device embodiments include a catheter with an inline needle, where the sharp tip of the needle is protectable by the catheter during use to prevent fistula/graft or peripheral vessel damage due to limb movement or needle migration. Exemplary vascular access catheter device embodiments may also function to prevent needle stick injuries to persons performing hemodialysis procedures, and may allow for parking of the catheter before final engagement, which permits minor adjustments to the catheter and the needle to obtain optimum blood flow.

Certain combined hemodialysis needle and catheter devices are known. In one such known device, a needle protrudes from a catheter portion of the device and is used to pierce a patient's skin so as to facilitate insertion of the needle tip and a portion of an overlying catheter into the vasculature (e.g., fistula/graft) of the patient. Once placed in the patient's fistula/graft, the needle portion is subsequently withdrawn by retracting a plunger to leave only the catheter inserted. Retracting the needle pulls it out of the catheter and into a housing portion of the device so as to prevent needle injury to the fistula/graft during use. However, since the needle is withdrawn completely from the catheter and into housing, the catheter alone is responsible for conducting blood flow during hemodialysis treatment. Unfortunately, because the catheter is typically thin-walled plastic tubing, it is often weak, which may lead to kinking and possible partial collapse due to high blood flow rate, thereby impairing the hemodialysis procedure.

Exemplary vascular access catheter device embodiments described and shown herein are single use devices designed to produce vascular access (i.e., fistula/graft or peripheral vessel access), and to connect to a dialysis machine via elongate tubing in the case of a hemodialysis application. An exemplary vascular access catheter device includes an inline needle that is concentrically located within a slidable catheter portion while protruding slightly therefrom during certain times. The sharp tip of the needle may be used to pierce a patient's skin and fistula/graft or peripheral vessel, whereafter a catheter tip of the slidable catheter portion will follow the needle tip into the fistula/graft or peripheral vessel and the catheter portion may subsequently be slidably displaced to cover the needle tip during use of the device. The catheter position relative to the needle position may be releasably or non-releasably secured to ensure that the needle tip remains within the catheter.

The catheter of an exemplary vascular access catheter device is thus placed without removing the needle, thereby maintaining the catheter and needle in communication with the patient's blood without a break in the dialysis blood flow path. The needle tip is also protected by the catheter during use of the device, which prevents fistula/graft/peripheral vessel injury and infiltration in patients and needle stick injuries to health care workers and other users of the device. Further, since the needle still resides within a majority of the catheter, issues such as kinking and partial collapse of the catheter are avoided, thereby preserving blood flow. Unlike known combination needle and catheter devices, exemplary vascular access catheter device embodiments do not require any special training prior to use.

An exemplary vascular access catheter device may include a needle portion having a needle that extends from a needle hub, and a catheter portion that is supported on the needle portion and includes a catheter that extends from a catheter hub. The needle may extend concentrically within the catheter such that a sharp tip of the needle protrudes from an open end of the catheter when the catheter portion is in a retracted position. The catheter portion is slidable relative to the needle portion, such that the catheter may be linearly displaced (extended) to cover the needle tip. An exemplary vascular access catheter device may also include a body portion that is supported on the needle portion. The body portion may include a gripping structure that facilitates handling and use of the device, and may be provided in the form of extending wings or in other forms that also facilitate removable attachment of the device to a patient during use.

In one exemplary vascular access catheter device, the position of the catheter portion relative to the needle portion of the device may be maintained by a retention mechanism in the form of a detent element on the catheter hub and an associated annular groove on the needle hub, or vice versa.

In another exemplary vascular access catheter device, the position of the catheter portion relative to the needle portion of the device may be maintained by a retention mechanism in the form of a bolt that extends from the needle hub through a detent groove in the catheter hub.

In another exemplary vascular access catheter device, the position of the catheter portion relative to the needle portion of the device may be maintained by a retention mechanism in the form of a tab that extends from the needle hub through a detent groove in the catheter hub.

In another exemplary vascular access catheter device, the position of the catheter portion relative to the needle portion of the device may be maintained by a retention mechanism in the form of a pin or bolt that extends from the needle hub through a curved cam slot in the catheter hub. The cam slot may allow for parking of the catheter portion in an extended position before final engagement with a patient.

In another exemplary vascular access catheter device, the position of the catheter portion relative to the needle portion of the device may be maintained by a retention mechanism in the form of a retention slot in the catheter hub that engages a gripping element of the needle portion when the catheter portion is extended. The retention slot may be designed to lock the catheter hub to the gripping element such that the catheter portion cannot be again retracted once fully extended.

An exemplary vascular access catheter device may include a seal between the needle and the catheter to prevent the leakage of blood from the device during use.

In at least one exemplary vascular access catheter device, the needle and catheter portion of the device may be enclosed within a safety cap when not in use.

The catheter hub and needle hub of an exemplary vascular access catheter device may be manufactured from a transparent or semi-transparent material to permit viewing of the flow of blood passing through the device during use.

Other aspects and features of the inventive concept will become apparent to those skilled in the art upon review of the following detailed description of exemplary embodiments along with the accompanying drawing figures.

### BRIEF DESCRIPTION OF THE DRAWINGS

In the following descriptions of the drawings and exemplary embodiments, like reference numerals across the several views refer to identical or equivalent features, and:

FIG. 1A is a top perspective view of one exemplary embodiment of a catheter device according to the general inventive concept, with a catheter portion thereof in a retracted position to expose a needle tip;

FIG. 1B is a bottom perspective view of the catheter device of FIG. 1A;

FIG. 1C is a side view of the catheter device of FIG. 1A;

FIG. 1D is a cross-sectional view of the catheter device of FIG. 1C;

FIG. 2A is a top perspective view of the catheter device of FIG. 1A, with the catheter portion thereof in an extended position to cover the needle tip;

FIG. 2B is a bottom perspective view of the catheter device of FIG. 2A;

FIG. 2C is a side view of the catheter device of FIG. 2A;

FIG. 2D is a cross-sectional view of the catheter device of FIG. 2C;

FIG. 3A depicts the catheter device of FIG. 1C with a protective cap;

FIG. 3B depicts the catheter device of FIG. 3A with aa section view of the protective cap;

FIGS. 4A-4B are a top perspective view and a side view, respectively, of another exemplary embodiment of a catheter device according to the general inventive concept, with a catheter portion thereof in a retracted position to expose a needle tip;

FIGS. 4C-4D are a top perspective view and a side view, respectively, showing the catheter device of FIGS. 4A-4B with the catheter portion thereof in an extended position to cover the needle tip;

FIG. 5A is a top perspective view of another exemplary embodiment of a catheter device according to the general inventive concept, with a catheter portion thereof in a retracted position to expose a needle tip;

FIG. 5B is an enlarged top plan and partial section view of a portion of the catheter device of FIG. 5A;

FIG. 5C shows the catheter device of FIG. 5A with the catheter portion in an extended position to cover the needle tip;

FIG. 5D is an enlarged top plan and partial section view of a portion of the catheter device of FIG. 5C;

FIG. 6A is a top perspective view of another exemplary embodiment of a catheter device according to the general inventive concept, with a catheter portion thereof in a retracted position to expose a needle tip;

FIG. 6B shows the catheter device of FIG. 6A with the catheter portion in an extended position to cover the needle tip;

FIGS. 7A-7C are side views of another exemplary embodiment of a catheter device according to the general inventive concept, wherein a catheter portion of the device is respectively shown in a fully retracted, partially extended and fully extended position;

FIGS. 8A-8B depict a perspective view and a cross-sectional side view, respectively, of an exemplary catheter portion of an exemplary embodiment of a catheter device according to the general inventive concept; and

FIGS. 8C-8D depict various possible features of an insertion end of an exemplary catheter of an exemplary catheter portion such as that shown in FIGS. 8A-8B.

### DETAILED DESCRIPTION OF THE EXEMPLARY EMBODIMENTS

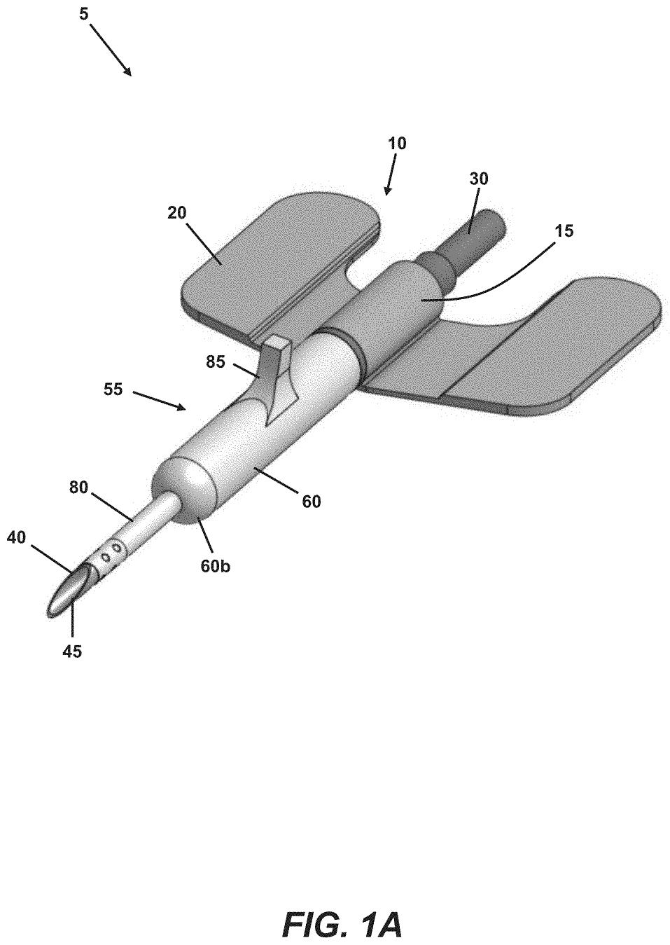

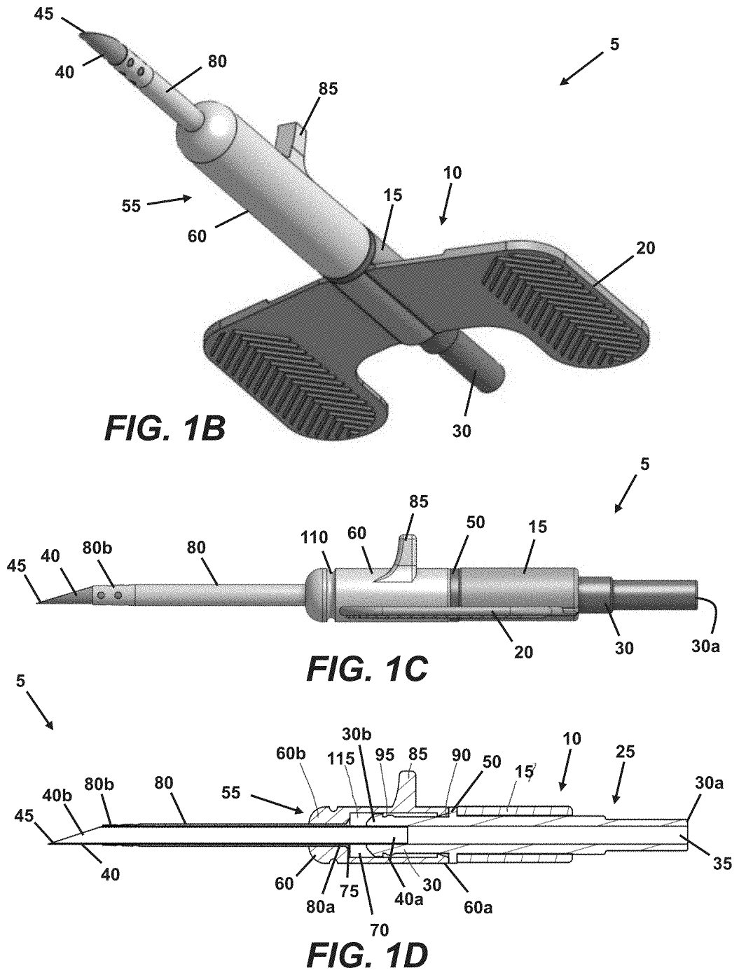

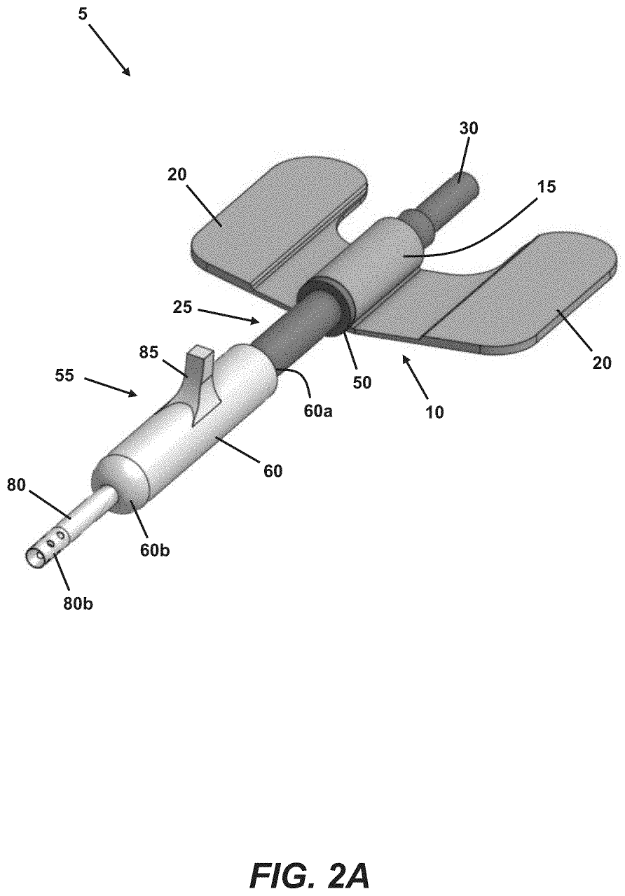

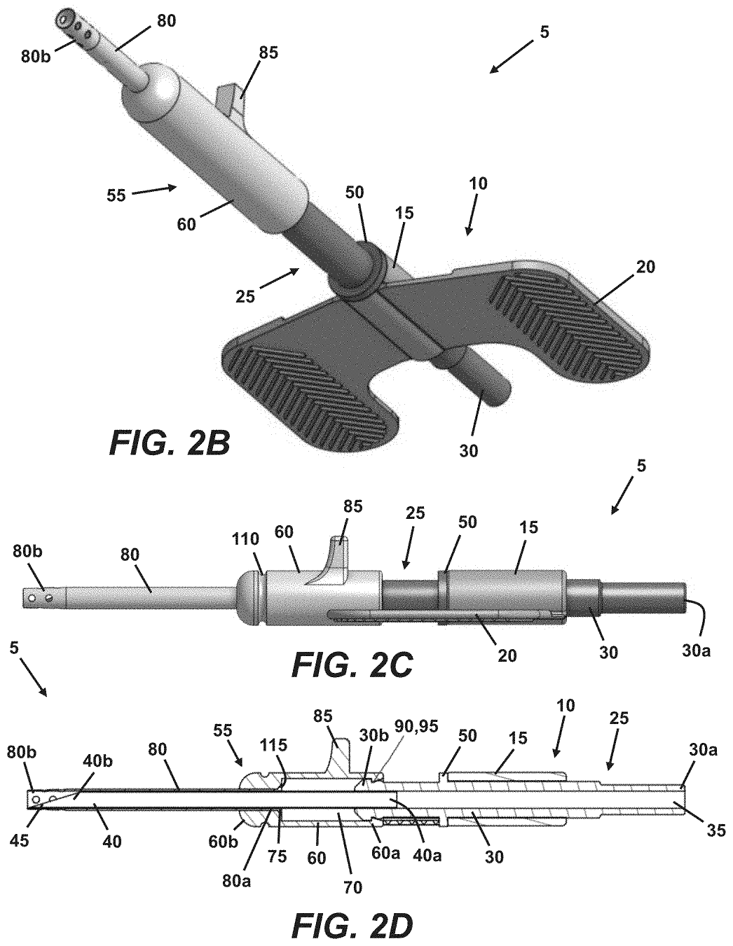

One exemplary embodiment of a vascular access catheter device (e.g., arteriovenous catheter device) 5 is illustrated in FIGS. 1A-2D. As may be observed, the device 5 includes a needle portion 25 on which is supported a body 10 and a catheter portion 55. The body 10 includes a central mounting sleeve 15 and a pair of wing-like gripping portions 20 that extend substantially laterally outward from opposite sides of the central mounting sleeve. While the overall body 10 may be of various shapes, in this exemplary embodiment the gripping portions 20 of the body are shaped as shown to facilitate gripping and manipulation of the device 10 by a user and taping of the device to a limb of a patient after fistula/graft or peripheral vessel access. Similarly, the central mounting sleeve 15 is cylindrical and hollow in the exemplary embodiment so as to slide over a correspondingly-shaped hub of a needle portion (see below), the mounting sleeve may be of other shapes as needed to cooperate with a given needle hub. The body 10 may be constructed from various different materials, with plastic (e.g., fluoroplastic) being a particularly good material.

It also may be observed that the catheter device 5 includes a needle portion 25 and a catheter portion 55. The needle portion 25 includes an elongate needle hub 30 having a proximal end 30a and a distal end 30b. An axial bore 35 passes through the needle hub to provide, among other things, a pathway for blood flow during a hemodialysis or other vascular access procedure. Tubing (not shown) may be coupled to the proximal end 30a of the needle hub for connecting the catheter device 5 to a hemodialysis machine, as would be understood by one of skill in the art.

A needle 40 of the needle portion 25 has a proximal end 40a thereof inserted sufficiently far into the bore 35 in the distal end 30b of the needle hub 30 to securely retain the needle. The proximal end 40a of the needle 40 may be retained in the needle hub 30 by a press fit or a similar interaction between the components. Alternatively, or in conjunction with such other techniques, an adhesive or other affixation mechanism may be used to further secure the needle 40 in the needle hub 30. A distal, free end 40b, of the needle is provided with a sharp point 45 for the purpose of piercing the skin of a patient and subsequently accessing the patient's fistula/graft or peripheral vessel. The specific needle gauge, needle construction, etc., may vary in keeping with accepted practices understood in the art.

The inside diameter of the central mounting sleeve 15 of the body 10 is dimensioned to pass over a portion of the needle hub 30. As shown, the needle hub 30 may include a protruding collar 50 or similar element that is designed and located to abut a distal face of the central mounting sleeve 15 and to thereby act as a stop for the body 10 when the body is properly installed on the needle hub 30. The collar may also act as a rear (retraction) stop for the catheter portion 55, as is described in more detail below.

The central mounting sleeve 15 of the body 10 may be retained on the needle hub 30 of the needle portion 25 by any of the same techniques mentioned above relative to securing the needle 40 in the bore 35 of the needle hub. Other techniques known to those of skill in the art may also be employed. Threaded engagement of the central mounting sleeve 15 and the needle hub 30 is also possible.

As shown particularly clearly in the section view of FIG. 1D, the collar 50 is located such that some length of the needle hub 30 extends distally therefrom. As is described below, this distally-extending portion of the needle hub 30 of this exemplary catheter device 5 is designed to retain and act as a guide for sliding movement of the catheter portion 55 of the device.

In similar fashion to the needle portion 25, the catheter portion 55 includes a catheter hub 60 with proximal and distal ends 60a, 60b. The size and shape of the catheter hub 60 is selected so that the catheter hub will cooperate with the needle hub 30. More specifically, the catheter hub 60 is of substantially cylindrical shape, and includes a first central bore 70 at the proximal end 60a that is dimensioned to permit the catheter hub to slide over the portion of the needle hub 30 that extends distally of the collar 50. This arrangement allows the catheter portion 55 of the catheter device 5 to be supported on the needle portion 25 and to slide linearly relative to the needle hub 30 (and the body 10). A second central bore 75 extends inward from the distal end 60b of the catheter hub 60 and opens into the proximally-located needle hub receiving bore 70.

The catheter portion 55 further includes a hollow catheter 80 that extends longitudinally outward from the distal end 60b of the catheter hub 60. In this exemplary embodiment, the catheter 80 has an open proximal end 80a that is inserted sufficiently far into the second bore 75 in the catheter hub 60 to securely retain the catheter. The proximal end 80a of the catheter 80 may be retained in the catheter hub 60 by any of the needle retention techniques mentioned above or by any other acceptable technique known to one of skill in the art. In other embodiments, the catheter 80 may be an integrally molded part of the catheter hub 60. A distal, free open end 80b, of the catheter 80 may be tapered and/or may include any other features that may facilitate entry of the free end of the catheter into the fistula/graft or peripheral vessel of the patient subsequent to initial access by the needle 40 and/or may facilitate blood flow during a hemodialysis or other vascular access procedure.

The inside diameter of the catheter is preferably similar in dimension to the outside diameter of the needle 40 so as to produce a close tolerance but sliding fit between the catheter 80 and the needle 40 when the catheter hub 60 is installed over the needle hub 30. Nonetheless, a seal 115 may be placed at or near the entry point of the second bore 75 into the first bore 70 to prevent possible blood leakage between the needle 40 and the catheter 80 during use of the catheter device 5. As with the needle 40, the specific construction of the catheter 80 may otherwise vary in keeping with accepted practices understood in the art. For example, the catheter may be constructed from a fluoroplastic material.

From the foregoing description and corresponding FIGS. 1A-2D, it should be understood that when the body 10 and catheter portion 55 are properly assembled to the needle portion 25, as explained above, the needle hub 30 is substantially concentrically located within the catheter hub 60, the needle 40 is substantially concentrically located within the catheter 80, and the catheter hub 60 and attached catheter 80 are together linearly slidable relative to the needle hub 30 and the attached needle 40. As shown in the drawing figures, a gripping element 85 or similar feature may be provided on the catheter hub 60 to facilitate sliding of the catheter portion 55 by a user of the device 5.

FIGS. 1A-1D show the catheter portion 55 in a retracted position, which results in the needle tip 45 and perhaps some additional length of the needle 40 protruding from the open distal end 80b of the catheter. This is the proper position in which the catheter portion 55 should reside during initial piercing of the patient's skin and accessing of the underlying fistula/graft or peripheral vessel using the needle 40 of the device 5.

With the tip of the needle 40 and the catheter 80 residing in the patient's fistula/graft or peripheral vessel, the catheter portion 55 may then be placed in an extended position (see FIGS. 2A-2D) by sliding the catheter portion linearly and in a distal direction along the needle hub 30 such that the sharp tip 45 of the needle becomes covered by the distal end 80b of the catheter 80. A distal catheter hub hard stop or a similar mechanism (see below) may be used to ensure proper linear positioning and possibly position retention of the catheter portion. With the needle 40 and the catheter 80 properly located in the fistula/graft or peripheral vessel of the patient, the catheter device 5 may be secured against movement, such as but not limited to by taping the gripping portions 20 of the body 10 to the limb of the patient.

As should be readily obvious to one of skill in the art, placing the catheter 80 of the exemplary catheter device 5 into the fistula/graft or peripheral vessel and over the needle tip 45 as described above serves to prevent the needle tip from damaging the fistula/graft or peripheral vessel and also, therefore, prevents infiltration and related hematoma problems. And unlike known devices, the needle 40 of the exemplary device 5 remains largely within the catheter 80 during a hemodialysis or other vascular access procedure, thereby substantially eliminating any chance that the catheter may collapse, kink, etc., and interfere with blood flow or cause damage on its own.

As described above, the catheter portion 55 has a retracted position where the catheter 80 is kept from interfering with use of the needle to initially access the fistula/graft or peripheral vessel of a patient. Likewise, the catheter portion 55 also has an extended position where the open distal end 80b of the catheter 80 will extend at least equal with if not beyond the tip 45 of the needle and into the fistula/graft or peripheral vessel currently accessed by the needle. Consequently, it is preferable to provide a mechanism by which it can be ensured that the catheter portion 55 is properly in the retracted position or the extended position, and by which either position can be maintained once selected.

In the case of the exemplary catheter device 5 of FIGS. 1A-2D, position retention is provided by a detent mechanism formed through interaction of certain elements of the catheter hub and the needle hub. This relationship may be reversed in other embodiments.

Referring to the sectional views of FIGS. 1D and 2D, it may be more specifically understood that abutting contact between the proximal end 60a of the catheter hub 60 and the distal side of the collar 50 of the needle hub 30 will act as a hard stop that can be used to set the proper retracted position of the catheter portion 55. It may also be observed that the exemplary catheter hub 60 of the exemplary catheter device 5 includes an inwardly-directed annular projection 90 at its proximal end 60a. The annular projection 90 may have a hook shape as shown, but other shapes are also possible. As explained further below, the annular projection 90 essentially acts as the lever element of the detent mechanism.

It may also be observed in FIGS. 1A and 2D that the needle hub 30 of the exemplary needle portion 25 is provided with an annular groove 95 near the distal end 30b thereof. The annular groove 95 essentially acts as the pawl element of the detent mechanism. As shown in FIG. 2D, when the catheter portion 55 is placed in the extended position, the annular projection 90 of the catheter hub 60 becomes releasably engaged with the annular groove 95 in the needle hub 30, thereby limiting the amount of possible linear movement and setting and retaining the extended position of the catheter portion 55, absent an overcoming displacement force provided by the user. Optionally, a second annular groove (not shown) may be similarly placed in the needle hub 30 in the area of the collar 50 to engage with the annular projection 90 of the catheter hub 60 and to releasably retain the catheter portion 55 in its retracted position absent an overcoming displacement force provided by the user.

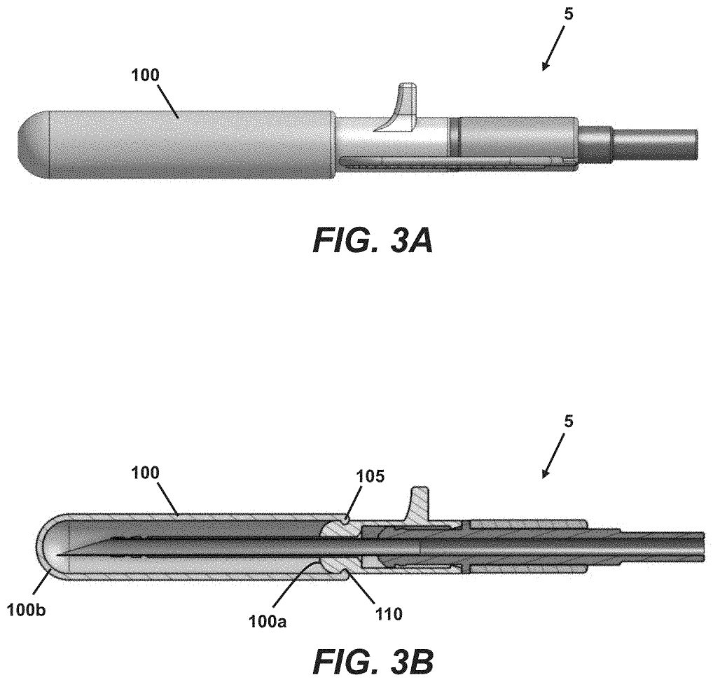

To guard against inadvertent contact with the sharp tip 45 of the needle 40, a protective device may be associated with the needle. As illustrated in FIGS. 3A-3B, a cap 100 having an open proximal end 100a and a closed distal end 100b may be provided in this exemplary catheter device embodiment to enclose the needle 40 and catheter and to protect users from accidental needle sticks. When such a cap is provided, the cap may vary in shape and size. Such a cap may also be removably retained on the catheter device in different ways. In this example, the open end of the cap is provided with an inwardly-projecting retention ridge 105 that is located and configured to engage a corresponding annular cap retention groove 110 (see e.g., FIG. 1C) provided in the catheter hub 60 near the distal end 60b thereof. Engagement of the retention ridge 105 with the cap retention groove 110 removably secures the cap 100 to the catheter hub 60.

Other cap retention techniques may be employed with other catheter device embodiments. Other exemplary catheter device embodiments may or may not include a cap.

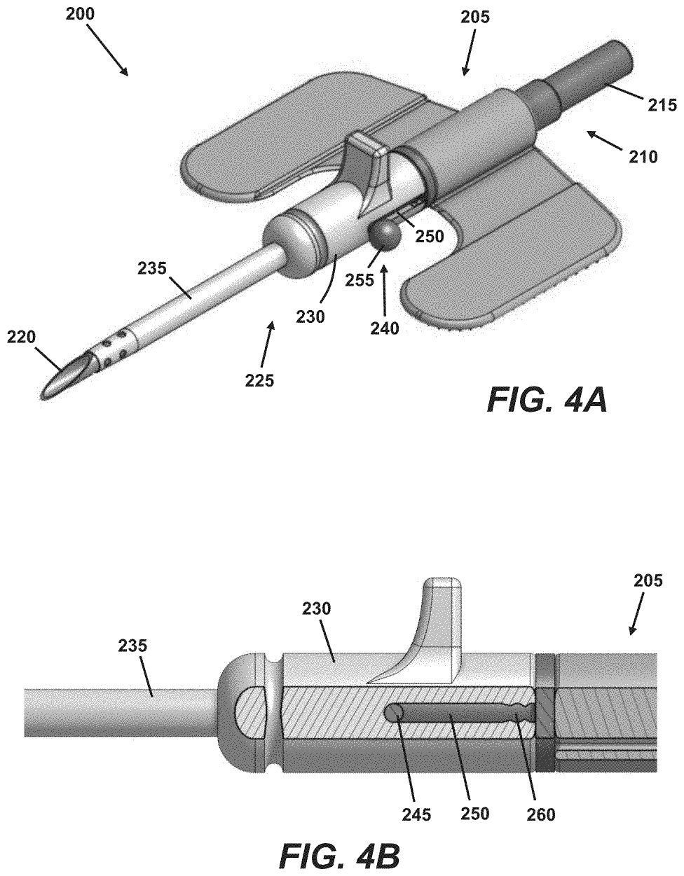

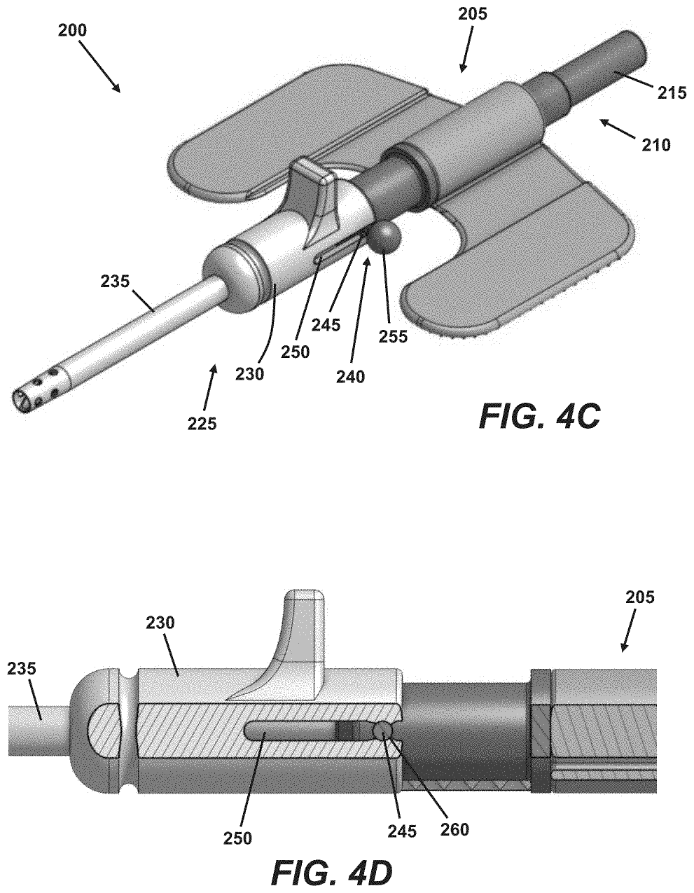

Other exemplary catheter device embodiments also may utilize catheter portion position retaining mechanisms that differ from the particular detent mechanism shown in FIGS. 1D and 2D and described above. One exemplary embodiment of such an alternative position retention mechanism is depicted in FIGS. 4A-4D.

The exemplary arteriovenous catheter device 200 depicted in FIGS. 4A-4D again includes a body 205 and a catheter portion 225 that are supported on a needle portion 210. The design and interrelationship of each of the needle portion 205 and the catheter portion 225 is generally as described above, as is the basic concept of using the device 200 in a hemodialysis operation. Thus, the needle portion 210 again includes a needle hub 215 and a needle 220. Likewise, the catheter portion 225 again includes a catheter hub 230 and a catheter 235.

In the case of the exemplary arteriovenous catheter device 200 shown in FIGS. 4A-4D, the catheter portion retention mechanism employs a bolt element 240 having a shaft portion 245 that extends from the needle hub 215 through a detent groove 250 in the catheter hub 230 to limit movement and retain the position of the catheter portion 225 of the device relative to the needle portion 210 of the device. The shaft 245 of the bolt element 240 may include an enlarged end portion 255 to ensure the bolt element shaft 245 and the detent groove 250 in the catheter hub 230 remain engaged.

As shown most clearly in FIG. 4B, the detent groove 250 may be configured such that the shaft 245 of the bolt element 240 is in contact with the closed end of the detent groove 250 when the catheter portion 225 is in a retracted position. Referring also to FIG. 4D, it can be further observed that the detent groove 250 may include one or more detent notches 260 that are releasably engageable with the shaft 245 of the bolt element 240 when the catheter portion 225 is in an extended position. The interaction between the detent notches 260 and the shaft 245 of the bolt element 240 serves to retain the catheter portion 225 in the extended position unless an overcoming displacement force is provided by the user.

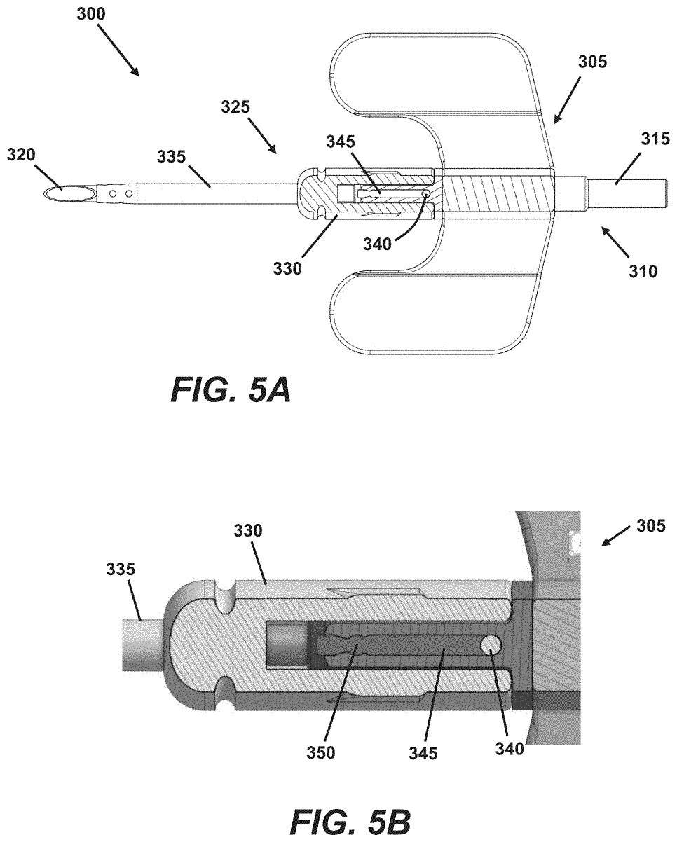

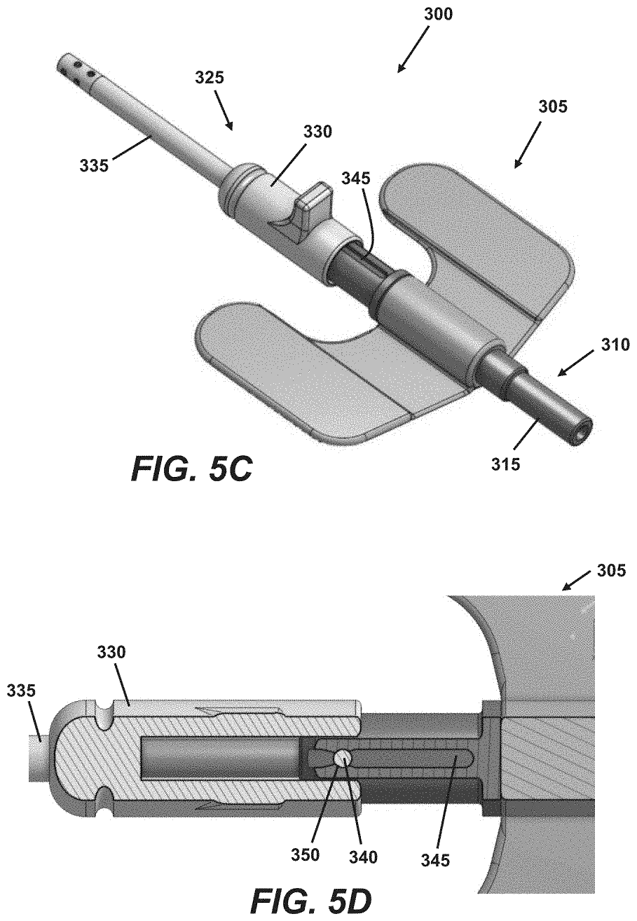

Another exemplary embodiment of an alternative catheter portion position retention mechanism is depicted in FIGS. 5A-5D. The exemplary arteriovenous catheter device 300 depicted in FIGS. 5A-5D again includes a body 305 and a catheter portion 325 that are supported on a needle portion 310. The design and interrelationship of each of the needle portion 305 and the catheter portion 325 is generally as described above, as is the basic concept of using the device 300 in a hemodialysis operation. Thus, the needle portion 310 again includes a needle hub 315 and a needle 320. Likewise, the catheter portion 325 again includes a catheter hub 330 and a catheter 335.

In the case of the exemplary arteriovenous catheter device 300 shown in FIGS. 5A-5D, the catheter portion retention mechanism employs a tab 340 that extends from the catheter hub 325 into a detent groove 345 in the needle hub 315 to limit movement and retain the position of the catheter portion 325 of the device relative to the needle portion 310 of the device. The tab 340 may be provided, for example, in the form of a pin.

As shown most clearly in FIG. 5B, the detent groove 345 may be configured such that the tab 340 is in contact with the closed end of the detent groove 345 when the catheter portion 325 is in a retracted position. Referring to FIG. 5D, it can also be observed that the detent groove 345 may include one or more detent notches 350 that are releasably engageable with the tab 340 when the catheter portion 325 is in an extended position. The interaction between the detent notches 350 and the tab 340 serves to retain the catheter portion 325 in the extended position unless an overcoming displacement force is provided by the user.

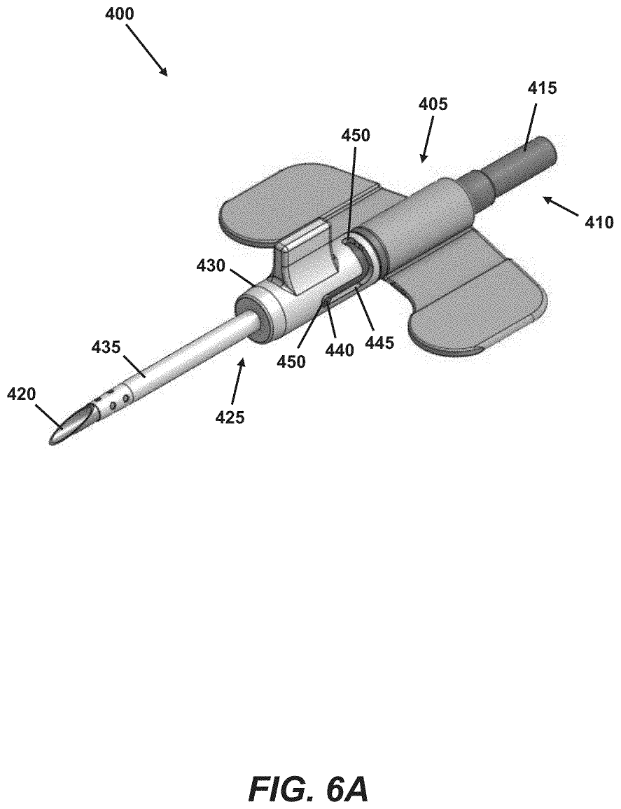

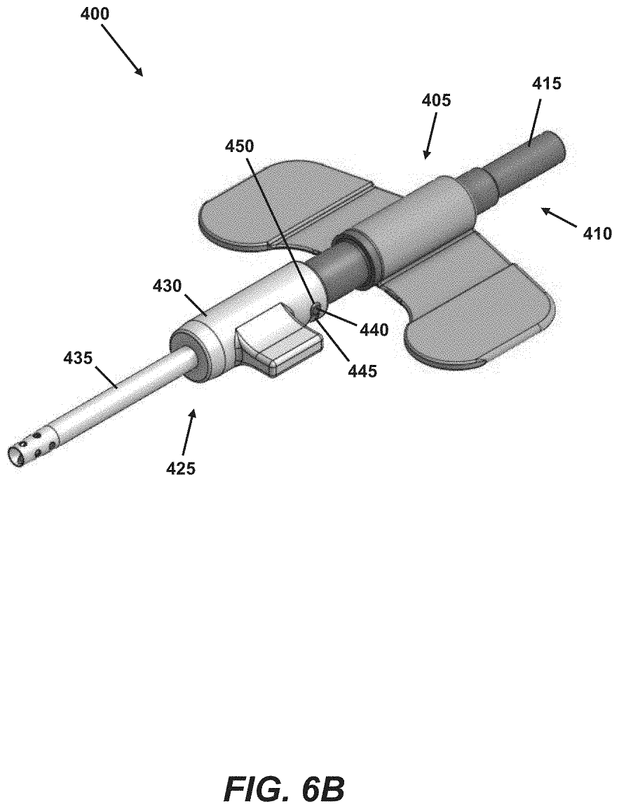

Another exemplary embodiment of an alternative catheter portion position retention mechanism is depicted in FIGS. 6A-6B. The exemplary arteriovenous catheter device 400 depicted in FIGS. 6A-6B again includes a body 405 and a catheter portion 425 that are supported on a needle portion 410. The design and interrelationship of each of the needle portion 410 and the catheter portion 425 is generally as described above, as is the basic concept of using the device 400 in a hemodialysis operation. Thus, the needle portion 410 again includes a needle hub 415 and a needle 420. Likewise, the catheter portion 425 again includes a catheter hub 430 and a catheter 435.

In the case of the exemplary arteriovenous catheter device 400 shown in FIGS. 6A-6B, the catheter portion retention mechanism employs a pin 440 that extends from the needle hub 415 through a curved cam slot 445 in the catheter hub 430 to limit movement and retain the position of the catheter portion 425 of the device relative to the needle portion 410 of the device. The cam slot 445 may be configured so as to wrap at least partially around the circumference of the catheter hub 430. As such, a linear displacement of the catheter hub 430 along the needle hub 415 will also produce a rotation of the catheter portion. During use of this exemplary arteriovenous catheter device 400, the cam slot 445 allows for parking of the catheter portion 425 in an extended position before final engagement, which permits minor adjustments to the catheter 435 and the needle 420 to obtain optimum blood flow.

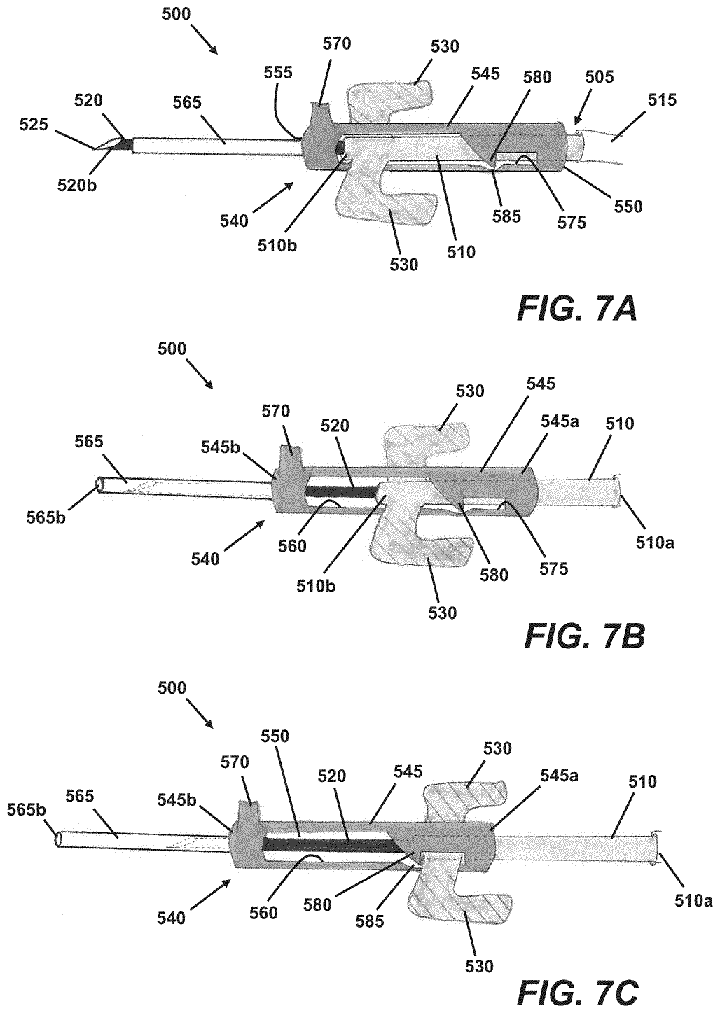

An exemplary embodiment of a vascular access catheter device 500 is illustrated in FIGS. 7A-7C. As may be observed, the device 500 includes a needle portion 505 and a catheter portion 540. The needle portion 505 includes an elongate needle hub 510 having a proximal end 510a and a distal end 510b. A hollow interior of the needle hub provides a pathway for blood flow during a hemodialysis procedure or when the device is otherwise used to access peripheral vessels. Tubing 515 may be coupled to the proximal end 510a of the needle hub 510 for connecting the catheter device 500 to a hemodialysis machine or to another device, as would be understood by one of skill in the art.

A needle 520 of the needle portion 505 has a proximal end (not visible) thereof that may be inserted sufficiently far into the bore at the distal end 510b of the needle hub 510 to securely retain the needle therein. The proximal end of the needle 520 may be retained in the needle hub 510 by a press fit or a similar interaction between the components. Alternatively, or in conjunction with such other techniques, an adhesive or other affixation mechanism may be used to further secure the needle 520 in the needle hub 510. A distal, free end 520b, of the needle is provided with a sharp point 525 for the purpose of piercing the skin of a patient and subsequently accessing a fistula/graft or peripheral vessel. The specific needle gauge, needle construction, etc., may vary in keeping with accepted practices understood in the art.

The needle portion 505 in this exemplary embodiment further includes a pair of wing-like gripping elements 530 that extend substantially laterally outward from opposite sides of the needle hub 510. While the gripping elements 530 may be of various shapes, in this exemplary embodiment the gripping elements 530 of the body are shaped as shown to facilitate gripping and manipulation of the device 500 by a user and taping of the device to a limb of a patient after fistula/graft or peripheral vessel access.

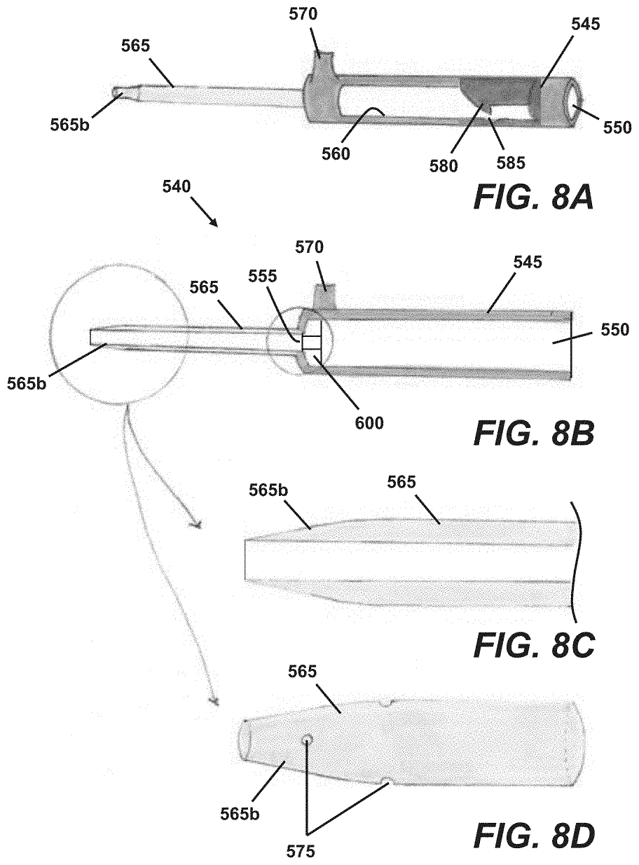

As previously described, the exemplary catheter device 500 also includes a catheter portion 540 that is supported on the needle portion 505 and includes a catheter hub 545 with proximal and distal ends 545a, 545b. The size and shape of the catheter hub 545 is selected so that the catheter hub will cooperate with the needle hub 510. More specifically, the catheter hub 545 is of substantially cylindrical shape, and includes a first central (needle hub-receiving) bore 550 at the proximal end 545a that renders the needle hub substantially hollow and is dimensioned to permit the catheter hub to slide over the needle hub 510. This arrangement allows the catheter portion 540 of the catheter device 500 to be supported on the needle portion 505 and for the catheter hub 540 and the needle hub 510 to slide linearly relative to one another as illustrated in FIGS. 7A-7C. A smaller diameter second central bore 555 (see also FIG. 8B) extends inward from the distal end 545b of the catheter hub 545 and opens into the needle hub receiving bore 550 to allow passage of the needle 520 through the distal end of the catheter hub 545.

The catheter hub 545 of this exemplary catheter device 500 further includes a pair of diametrically opposed gripping element relief slots 560, through which the gripping elements 530 of the needle portion 505 may protrude when the catheter portion 540 is properly installed over the needle portion 505. If an exemplary catheter device embodiment utilizes only a single gripping element 530, the catheter hub 545 may, but is not required to, include only one corresponding gripping element relief slot 560.

The catheter portion 540 further includes a hollow catheter 565 that extends longitudinally outward from the distal end 545b of the catheter hub 545 and includes open proximal and distal ends. In some embodiments, the proximal end (not visible) of the catheter 565 may be inserted sufficiently far into the second central bore 555 in the catheter hub 545 to securely retain the catheter therein. In such an embodiment, the proximal end of the catheter 565 may be retained in the catheter hub 545 by any of the needle retention techniques mentioned above or by any other acceptable technique known to one of skill in the art. In other embodiments, the catheter 565 may be an integrally molded part of the catheter hub 545. A distal, free open end 565b, of the catheter 565 may be tapered and/or may include any other features that may facilitate entry of the free end of the catheter into the fistula/graft or peripheral vessel of the patient subsequent to initial access by the needle 520 and/or may facilitate blood flow during a hemodialysis or other vascular access procedure.

The inside diameter of the catheter 565 is preferably similar in dimension to the outside diameter of the needle 520 so as to produce a close tolerance sliding fit between the catheter 565 and the needle 520 when the catheter hub 545 is installed over the needle hub 510. Nonetheless, a seal 600 (see FIG. 8B) may be placed at or near the entry point of the second bore 555 into the first bore 550 to prevent possible blood leakage between the needle 520 and the catheter 565 during use of the catheter device 500. As with the needle 520, the specific construction of the catheter 565 may otherwise vary in keeping with accepted practices understood in the art. For example, the catheter 565 may be constructed from a fluoroplastic material.

From the foregoing description and corresponding FIGS. 7A-7C, it should be understood that when the catheter portion 540 is properly assembled to the needle portion 505, as explained above, a portion of the needle hub 510 is substantially concentrically located within the catheter hub 545, a portion of the needle 520 is substantially concentrically located within the catheter 565, and the catheter hub 545 and attached catheter 565 are together linearly slidable relative to the needle hub 510 and the attached needle 520. As shown in the drawing figures, a grip 570 or similar feature may be provided on the catheter hub 545 to facilitate sliding of the catheter portion 540 by a user of the device 500.

FIG. 7A shows a position where the needle portion 505 is fully extended relative to the catheter portion 540 (or the catheter portion is fully retracted relative to the needle portion), which results in the needle tip 525 and perhaps some additional length of the needle 520 protruding from the open distal end 565b of the catheter. This is the proper positioning of the needle portion 505 and the catheter portion 540 for initial piercing of the patient's skin and accessing of the underlying fistula/graft or peripheral vessel using the needle 520 of the catheter device 500.

After the tip of the needle 520 has been inserted into the patient's fistula/graft or peripheral vessel, the catheter portion 540 may be moved toward an extended position (see FIGS. 7A-7C) by sliding the catheter portion linearly and in a proximal-to-distal direction along the needle hub 510 such that the sharp tip 525 of the needle 520 becomes covered by the distal end 565b of the catheter 565 and a portion of both the needle and the catheter temporarily reside in the fistula/graft or peripheral vessel. The grip 570 on the catheter hub 545 may be used to facilitate linear movement of the catheter portion 540.

Once blood flash is seen in the catheter 565, the needle portion 505 may be slid slightly in a proximal or distal direction to move the needle 520 relative to the catheter 565 and to resultantly achieve optimum blood flow and position. The needle portion 505 may then be “parked” in this position, but the positional relationship of the catheter 565 and needle 520 may also be further adjusted if needed to maintain an optimized blood flow. Once the final desired position of the catheter 565 and optimized blood flow is achieved, the needle portion 505 is retracted (such as by using the gripping elements 530) relative to the catheter portion 540 until travel of the needle portion is halted by a hard stop and the needle portion reaches an irreversible position (as explained below). With the catheter 565 properly located in the fistula/graft or peripheral vessel of the patient and the needle portion 505 placed in the irreversible retracted position, the catheter device 500 may be secured to the limb of the patient using tape or another technique known in the art.

As should be readily obvious to one of skill in the art, placing the catheter 565 of the exemplary catheter device 500 into the fistula/graft or peripheral vessel and over the needle tip 525 as described above serves to prevent the needle tip from damaging the fistula/graft or peripheral vessel and also, therefore, prevents infiltration and related hematoma problems. And unlike known devices, the needle 520 of the exemplary catheter device 500 remains partially within the catheter 565 during a hemodialysis or other vascular procedure, thereby substantially eliminating any chance that the catheter may collapse, kink, etc., and interfere with blood flow or cause damage on its own.

As described above, the catheter portion 540 has a retracted position where the catheter 565 is kept from interfering with use of the needle 520 to initially access the fistula/graft or peripheral vessel of a patient. Likewise, the needle portion 505 has an extended position that corresponds to the retracted position of the catheter portion 540 and a retracted position where the needle 520 is drawn into the catheter 565 and out of the fistula/graft or peripheral vessel once the catheter is inserted therein and blood flow has been optimized. Consequently, in addition to securing the overall catheter device 500 to the patient, it is also preferable to provide mechanisms by which it can be ensured that the position of the catheter portion 540 relative to the position of the needle portion 505 will be maintained after the needle 520 has been fully retracted.

As may be understood from FIG. 7A, contact between the distal end 510b of the needle hub 510 and the interior wall of the distal end of the catheter hub 545, and/or contact between a leading edge of the gripping elements 530 and a distal terminus of the gripping element relief slots 560 may act as a hard stop and set the fully extended position of the needle portion 505 and the fully retracted position of the catheter portion 540.

Referring to FIGS. 7B-7C, it may be observed that in the case of the exemplary catheter device 500, a needle portion retraction hard stop and needle portion retracted position retention functionality are both provided via interaction of certain elements of the catheter hub 545 and the needle hub 510. More specifically, the proximal end of at least one of the gripping element relief slots 560 in the catheter hub 545 leads into a needle portion retention mechanism having a retention slot 575 that extends further toward the proximal end 545a of the catheter hub.

The needle portion retention mechanism also includes a retention tab 580—which may be an integral portion of the catheter hub 545. The retention tab 580 protrudes into the retention slot 575 near a distal end thereof. The retention tab 580 may be shaped to facilitate passage of the corresponding needle portion gripping element 530—which also comprises an element of the needle portion retention mechanism—when the needle portion is moved in a distal-to-proximal direction relative to the catheter portion 540 toward its fully retracted position, but to prevent passage of the gripping element upon a subsequent attempt to thereafter move the needle portion in a proximal-to-distal direction back toward its extended position. For example, and as shown, the retention tab 580 may have a distal (leading) edge that slopes toward the proximal end 545a of the catheter hub 545, but a proximal (trailing) edge that is substantially parallel to the proximal end of the catheter hub (i.e., substantially transverse to the central axis of the device 500). Other retention tab trailing edge shapes may also be possible, as long as the trailing edge of the retention tab 580 is operative to engage the leading edge of the gripping element 530 and to prohibit an extending motion of the needle portion 505 once the needle portion has reached its fully retracted position.

The retention tab 580 may be aligned with a recess 585 or similar contour in the retention slot 575 to facilitate passage of the gripping element 530 past the retention tab 580 during an extending movement of the catheter portion 540. Passage of the gripping element 530 past the retention tab 580 during an extending movement of the catheter portion 540 may be instead or further facilitated by some controlled degree of flexibility of the retention tab 580 and/or the gripping element 530. For example, it will be common for a user to bend the gripping elements 530 when displacing the needle portion 505 during insertion or retraction of the needle 520 and to return the gripping elements to a substantially flat position before taping the catheter device 500 to a limb of a patient. Such a bending of the gripping elements 530 may also assist in passage of the gripping elements past the retention tab 580 of the retention mechanism during retraction of the needle portion 505.

As may be best observed in FIG. 7C, the position of the retention tab 580 and the size and shape of the portion of the retention slot 575 that resides proximally of the retention tab is such to accommodate a corresponding portion of the gripping element 530. More particularly, said portion of the retention slot 575 is preferably of like shape and only slightly larger than the corresponding portion of the gripping element 530 so as to trap the corresponding portion of the gripping element 530 between the trailing edge of the retention tab 580 and a proximal terminus of the retention slot 575, and to minimize or prohibit any further linear movement of the catheter portion 540 relative to the needle portion 505. Abutting contact between the proximal terminus of the retention slot 575 and a proximal edge of the gripping element 530 also functions as a hard stop for the fully retracted position of the needle portion 505. Likewise, the size and shape of the portion of the retention slot 575 that resides proximally of the retention tab also substantially prevents any rotation of the catheter portion 540 relative to the needle portion 505 when the needle portion is in a fully retracted position.

For further reference and clarity, a perspective view and a cross-sectional side view of the exemplary catheter portion 540 of the exemplary catheter device 500 of FIGS. 7A-7C is shown in FIGS. 8A and 8B, respectively. As previously stated, the dimensions of a given catheter device according to the inventive concept may vary as needed. However, in the exemplary embodiment of the device 500 of FIGS. 7A-7C, the length of the catheter hub 540 may be about 38 mm; the outside diameter of the catheter hub may be about 5.6 mm; the inside diameter (bore 550) of the catheter hub may be about 4 mm (for a wall thickness of about 0.8 mm); the length of the gripping element relief slot(s) 560 in the catheter hub may be about 17 mm; the length of the catheter 565 may be about 17 mm; the inside diameter of the catheter may be about 1.9 mm; and the catheter may have a wall thickness of about 0.25 mm.

As further illustrated in FIGS. 8C-8D, the catheter 565 may have a tapered distal end 565b to facilitate insertion into the hole in a patient's skin and fistula/graft or peripheral vessel created by the leading end of the needle 520. A plurality of orifices 575 may also be distributed about the distal end 565b of the catheter 565 to optimize blood flow into the catheter. For example, four diametrically opposed smooth orifices 575 may be present and each orifice may have a diameter of about 0.7 mm.

The material(s) used to produce the exemplary device 500 may also vary. For example, parts of the device 500 other than the needle 520 may be comprised of one or more thermoplastic polymers. In this regard, it has been found that manufacturing the catheter hub 545 from a polycarbonate, polyethylene or polypropylene material provides good results, as does manufacturing the catheter from polyethylene, polypropylene or flouroethylenepropylene (FEP). The needle 520 may be comprised of an acceptable metallic material or of any other material that is currently known to be acceptable or hereafter becomes acceptable in the art.

Various exemplary vascular access catheter device embodiments have been described and shown herein for purposes of illustration. Other variations are also possible. For example, and without limitation, the catheter hub and needle hub of an exemplary vascular access catheter device, may be manufactured from a transparent or semi-transparent material to permit viewing of the flow of blood passing through the device during use.

As used herein, the term “distal” is intended to refer to the end of the catheter device where the needle normally resides, and the term “proximal” is intended to refer to the end of the catheter device where fluid transport tubing is normally connected.

As used herein, the terms “axial” or “axially” are intended to refer to a direction that is parallel to the length-wise axis of the element/component to which the term is being applied.

As used herein, the term “central axis” is intended to refer to the symmetrical axis of a component or the device, and the term “central bore” is intended to refer to a bore that is symmetrical about the central axis.

As used herein “first” and “second” are intended only to differentiate between two elements or components for purposes of description, and not to indicate an order, a preference, or superiority or inferiority, of any kind.

As used herein, the term “irreversible” is intended to mean not able to be reversed under normal operation or when subjected to normal operating forces, and does not necessarily require that reversal is impossible under other conditions.

As used herein, the term “prohibit” is intended to mean prevent or make impossible under normal operation or when subjected to normal operating forces, and does not necessarily require that prohibition is impossible under other conditions.

While certain embodiments of the inventive concept are described in detail above, the scope of the inventive concept is not considered limited by such disclosure, and modifications are possible without departing from the spirit of the inventive concept as evidenced by the following claims:

What is claimed is:

1. A vascular access catheter device with an inline needle, comprising:
a catheter portion having an elongate hollow catheter hub and a catheter that extends from a distal end of the catheter hub;
a longitudinally-oriented relief slot through a wall of the catheter hub;
a needle portion having an elongate hollow needle hub, a needle that extends from a distal end of the needle hub, and a gripping element that extends transversely outward from the needle hub;
wherein, at least the distal end of the needle hub is arranged within the catheter hub such that the gripping element of the needle portion extends through the relief slot in the catheter hub, and the needle portion is selectively linearly displaceable within the catheter hub between an extended and a retracted position whereby a tip of the needle will respectively protrude from or reside within the catheter.

2. The vascular access catheter device of claim 1, wherein a proximal end of the needle hub is configured for the connection of hemodialysis or other fluid transport tubing.

3. The vascular access catheter device of claim 1, wherein contact between a distal edge of the gripping element and a distal terminus of the catheter hub relief slot functions as a needle portion extending movement stop mechanism.

4. The vascular access catheter device of claim 1, wherein contact between a distal end of the needle hub and an interior wall of the distal end of the catheter hub functions as a needle portion extending movement stop mechanism.

5. The vascular access catheter device of claim 1, further comprising a needle portion retracted position retention mechanism located at a proximal terminus of the catheter hub relief slot and configured to retain the needle portion in a fully retracted position.

6. The vascular access catheter device of claim 5, wherein the needle portion retracted position retention mechanism comprises:
a longitudinally-oriented retention slot in the catheter hub that extends from the proximal terminus of the catheter hub relief slot toward the proximal end of the catheter hub; and
a portion of the gripping element that is configured to be retained within the retention slot when the needle portion is placed in the fully retracted position.

7. The vascular access catheter device of claim 6, further comprising a retention tab on the catheter hub that extends into the retention mechanism retention slot and is operative to prohibit proximal-to-distal movement of the needle portion after the needle portion is placed in the fully retracted position.

8. The vascular access catheter device of claim 7, wherein the retention tab has a distal edge that slopes toward the proximal end of the catheter hub to facilitate passage of the gripping element during distal-to-proximal movement of the needle portion, and a proximal edge that is configured to resist passage of the gripping element during an attempted proximal-to-distal movement of the needle portion from the fully retracted position.

9. The vascular access catheter device of claim 6, wherein contact between a proximal edge of the gripping element and a proximal terminus of the retention mechanism retention slot functions as a needle portion retracting movement stop mechanism.

10. The vascular access catheter device of claim 9, wherein the needle portion retracting movement stop mechanism determines the fully retracted position of the needle portion.

11. The vascular access catheter device of claim 1, wherein at least the distal end of the needle hub is substantially concentrically arranged within the catheter hub, and at least a portion of the needle is substantially concentrically arranged within the catheter.

12. The vascular access catheter device of claim 1, wherein the catheter and needle are insertable into the bloodstream of a patient without a break in an overall dialysis blood flow path.

13. The vascular access catheter device of claim 1, wherein the gripping element exhibits at least some degree of flexibility.

14. The vascular access catheter device of claim 1, further comprising a seal between the needle and the catheter to prevent the leakage of blood from the device during use.

15. A vascular access catheter device with an inline needle, comprising:
a catheter portion having an elongate hollow catheter hub and a catheter that extends from a distal end of the catheter hub;
a pair of longitudinally-oriented and diametrically-opposed relief slots in the catheter hub;
a needle portion having an elongate hollow needle hub having a proximal end configured for the connection of hemodialysis or other fluid transport tubing, a needle that extends from a distal end of the needle hub, and a pair of diametrically-opposed gripping elements extending transversely outward from the needle hub, at least the distal end of the needle hub being concentrically arranged within the catheter hub such that the gripping elements extend through the relief slots in the catheter hub and at least a portion of the needle is concentrically arranged within the catheter;
a seal between the needle and the catheter to prevent the leakage of blood from the device during use; and
a needle portion retracted position retention mechanism located at a proximal terminus of at least one of the catheter hub relief slots and configured to retain the needle portion in a fully retracted position;
wherein the needle portion is selectively linearly displaceable within the catheter hub between an extended position where a tip of the needle will protrude from an open distal end of the catheter and the fully retracted position where the tip of the needle will reside within the catheter.

16. The vascular access catheter device of claim 15, further comprising a needle portion extending movement stop mechanism selected from the group consisting of contact between a distal edge of the gripping element and a distal terminus of the catheter hub and contact between a distal end of the needle hub and an interior wall of the distal end of the catheter hub.

17. The vascular access catheter device of claim 15, wherein the needle portion retracted position retention mechanism comprises:
a longitudinally-oriented retention slot in the catheter hub that extends from the proximal terminus of at least one of the catheter hub relief slots toward the proximal end of the catheter hub;
a portion of the corresponding gripping element that extends through the corresponding retention slot when the needle portion resides in the fully retracted position; and
a retention tab on the catheter hub that projects into the retention slot, the retention tab configured to facilitate distal-to-proximal movement of the gripping element past the retention tab but to prohibit subsequent proximal-to-distal movement of the gripping element and associated needle portion.

18. The vascular access catheter device of claim 17, wherein contact between a proximal edge of the gripping element and a proximal terminus of the retention mechanism retention slot functions as a needle portion retracting movement stop mechanism that determines the fully retracted position of the needle portion.

19. A method of safely performing hemodialysis on a patient with a fistula/graft, comprising:
(a) providing an arteriovenous catheter device with an inline needle, the arteriovenous catheter device comprising:
a catheter portion having an elongate hollow catheter hub and a catheter that extends from a distal end of the catheter hub,
a longitudinally-oriented relief slot through a wall of the catheter hub,
a needle portion having an elongate hollow needle hub, a needle that extends from a distal end of the needle hub, and a gripping element that extends transversely outward from the needle hub, at least the distal end of the needle hub arranged within the catheter hub such that the gripping element of the needle portion extends through the relief slot in the catheter hub, and the needle portion is selectively linearly displaceable within the catheter hub between an extended position where the tip of the needle will protrude from the catheter and a retracted position where the tip of the needle will reside within the catheter,
a needle portion retracted position retention mechanism located at a proximal terminus of the catheter hub relief slot and configured to retain the needle portion in a fully retracted position, and
hemodialysis tubing connected to a proximal end of the needle hub;
(b) piercing the skin of the patient with the tip of the arteriovenous catheter device needle and thereafter inserting the needle into the underlying fistula/graft to initiate blood flow through the arteriovenous catheter device;
(c) extending the catheter portion of the arteriovenous catheter device relative to the needle portion so that an open distal end of the catheter enters the fistula/graft and the tip of the needle becomes covered by the catheter;
(d) retracting the needle portion of the arteriovenous catheter device to the fully retracted position while a portion of the catheter remains in the fistula/graft of the patient; and
(e) releasably securing the arteriovenous catheter device to the patient.

20. The method of claim 19, further comprising adjusting the position of the arteriovenous catheter device catheter within the fistula/graft of the patient to optimize blood flow before retracting the needle portion of the arteriovenous catheter device.
